(19) United States
(12) Patent Application Publication
(10) Pub. No.: US 2025/0152772 A1
(43) Pub. Date: May 15, 2025

(54) PHOTOPOLYMERISABLE COMPOSITION FOR ADHESIVE FOR BIOLOGICAL TISSUES

(71) Applicant: COHESIVES, DIJON (FR)

(72) Inventors: Bertrand PERRIN, DIJON (FR); Natacha GOUTAY, DIJON (FR)

(73) Assignee: COHESIVES, DIJON (FR)

(21) Appl. No.: 18/839,052

(22) PCT Filed: Feb. 17, 2023
(86) PCT No.: PCT/FR2023/050229

§ 371 Date: Aug. 16, 2024

(30) Foreign Application Priority Data
Feb. 18, 2022 (FR) FR2201481

(51) Int. Cl. A61L 24/04; A61L 15/58

(52) U.S. Cl. CPC A61L 24/043; A61L 15/58

(57) ABSTRACT

A polymerisable composition for use as an adhesive for biological tissue, in particular for non-mineralised biological tissue, including an acrylate-type polymerisable monomer, a photoinitiator and a photopolymerisable resin. In particular, the composition of the invention makes it possible, after polymerisation, to adhere pieces of biological tissue to each other, to adhere a material to biological tissue, to form a surgical sealant or to form a skin dressing.

### FIELD OF THE INVENTION

The invention relates to a polymerisable composition, for use as an adhesive for biological tissues, in particular for non-mineralised biological tissues. The composition of the invention enables in particular, after polymerisation, the adhesion of biological tissues to one another, the adhesion of a material, a glue or a substance to a biological tissue, the formation of a surgical sealant or the formation of a skin dressing.

### PRIOR ART

Adhesives for conventional dressings, in particular skin dressings, do not tolerate humidity well, which leads to their debonding. The humidity can come from exudates of the wound, from sweat or from daily activities.

Medical adhesives are also of interest for replacing or facilitating sutures and to replace dressings. The application of medical adhesives is most often made directly on the tissue, without preparation of the bonding surface. Currently available medical adhesives have adhesive properties which are sometimes insufficient and/or have poor stability over time.

Furthermore, medical adhesives can be used in a certain number of surgical techniques. However, the efficiency of currently available surgical glues is controversial.

There is therefore a need to improve the properties of medical adhesives.

Medical adhesives can be in the form of polymerisable compositions. The polymerisation can be carried out, for example, under the action of the humidity of the tissues on which the composition is applied, or by external chemical or physical initiation, such as for example under the effect of UV or visible radiation.

In order to improve the properties of medical adhesives, compositions comprising a polymerisable monomer and having low viscosities have been proposed (WO2016/185153). These compositions penetrate more easily into the tissues, which makes it possible to obtain a better quality of bonding due to an anchoring of the adhesive in the tissue after polymerisation. In certain indications, these bondings may need to be combined with an additional adhesive in order to improve the properties. Moreover, these compositions are difficult for the general public to use directly.

The present invention proposes providing a new type of medical adhesive enabling these various problems to be overcome. The compositions according to the invention comprise a polymerisable acrylate monomer, a photopolymerisable resin and a photoinitiator.

The compositions according to the invention make it possible to obtain suitable adhesion, both for surgical applications as well as for skin adhesion for the general public.

The concentrations of the components of the composition of the invention enable a rapid initiation and polymerisation in several tens of seconds, without causing tissue burns due to a controlled exothermy.

Moreover, the combined presence of a photopolymerisable resin, with the photoinitiator and the polymerisable monomers used, gives the bonding obtained properties of flexibility and conformability suitable for soft biological tissue, good stability over time under the action of physiological liquids (e.g. blood, exudates, transpiration), prevents the accumulation of transpiration or exudates and provides good resistance to water.

### SUMMARY

The present invention therefore relates to a polymerisable composition, for use as an adhesive for non-mineralised biological tissues, comprising:
5% to 60% by weight, relative to the total weight of the composition, of a polymerisable monomer comprising an acrylate, methacrylate, acrylamide or methacrylamide function and not comprising a phosphate or phosphonate function;
0.1% to 5% by weight of a photoinitiator; and
a photopolymerisable resin;
said composition not comprising a monomer comprising a phosphate or phosphonate function on the one hand and an acrylate, methacrylate, acrylamide or methacrylamide function on the other hand.

According to an embodiment, the polymerisable monomer is chosen from: acrylic acid (AA), tert-butyl acrylate (tBuA), 2-hydroxyethyl methacrylate (HEMA), methacrylic acid (MA), lauryl acrylate (LA), lauryl methacrylate (LMA), 2-ethoxyethyl methacrylate (2EEMA), di(ethylene glycol) ethyl ether acrylate (DEGEA), 2-phenyloxyethyl acrylate (2PEA), 2-ethylhexyl methacrylate (2EHMA), n-butyl acrylate (nBuA), isobornyl acrylate (IBOA), isobornyl methacrylate (IBOMA), cyclic trimethylolpropane formal acrylate (CTFA), 3,3,5 trimethyl cyclohexanol methacrylate, tert-butyl methacrylate (tBuMA), methyl acrylate (MeA), methyl methacrylate (MMA), 2-ethylhexyl acrylate (2EHA), 2-(dimethylamino)ethyl acrylate (DAEA), 3-sulfopropyl acrylate potassium salt (SAPS), 3,3-dimethacrylic acid (DAA), crotonic acid (CA), triethylene glycol methyl ether methacrylate (TEGMEMA), 2-phenyloxyethyl methacrylate (2PEMA), 2-hydroxyethyl acrylate (HEA), 3-(trimethoxysilyl) propyl methacrylate, ethyl acrylate (EtA), cyclohexyl methacrylate, 3-hydroxypropyl acrylate, alpha-bromoethyl acrylate, alpha-chloroethyl acrylate, chloromethyl methacrylate, 2-bromoethyl methacrylate, 2-naphtyl methacrylate, paratolyl acrylate, parachlorophenyl methacrylate, metabromophenyl acrylate, 2,4,6-tribromophenyl acrylate, parachlorobenzyl methacrylate, metamethoxybenzyl methacrylate, paraethylbenzyl acrylate, 1,6-hexanediol dimethacrylate, neopentylglycol diacrylate, thiodiethylene-glycol dimethacrylate, bisphenol A ethoxyl diacrylate, bisphenol A ethoxyl dimethacrylate, pentaerythritol triacrylate, glyceryl triacrylate, dipentaerythritol pentaacrylate, trimethylolpropane triacrylate, tris(2-hydroxyethyl) isocyanurate trimethacrylate, trimethylolpropane polyoxyethylene triacrylate, urethane acrylate, urethane methacrylate, bis(4-methacryloylthiophenyl) sulfide, ethyleneglycol acrylate, polyethyleneglycol acrylate, ethyleneglycol methacrylate, polyethyleneglycol methacrylate, ethyleneglycol diacrylate, polyethyleneglycol diacrylate, ethyleneglycol dimethacrylate, polyethyleneglycol dimethacrylate, and the mixtures thereof.

According to an embodiment, the polymerisable monomer is chosen from: acrylic acid (AA), tert-butyl acrylate (tBuA), 2-hydroxyethyl methacrylate (HEMA), methacrylic acid (MA), lauryl acrylate (LA), lauryl methacrylate (LMA), 2-ethoxyethyl methacrylate (2EEMA), di(ethylene glycol) ethyl ether acrylate (DEGEA), 2-phenyloxyethyl acrylate (2PEA), 2-ethylhexyl methacrylate (2EHMA), n-butyl acrylate (nBuA), isobornyl acrylate (IBOA), isobornyl methacrylate (IBOMA), cyclic trimethylolpropane formal acrylate (CTFA), 3,3,5 trimethyl cyclohexanol methacrylate, tert-butyl methacrylate (tBuMA), methyl acrylate (MeA), methyl methacrylate (MMA), 2-ethylhexyl acrylate (2EHA), 2-(dimethylamino)ethyl acrylate (DAEA), 3-sulfopropyl acrylate potassium salt (SAPS), 3,3-dimethacrylic acid (DAA), crotonic acid (CA), triethylene glycol methyl ether methacrylate (TEGMEMA), 2-phenyloxyethyl methacrylate (2PEMA), 2-hydroxyethyl acrylate (HEA), 3-(trimethoxysilyl) propyl methacrylate, and the mixtures thereof.

According to an embodiment, the polymerisable monomer is at a concentration ranging from 10% to 40% by weight relative to the total weight of the composition.

According to an embodiment, the photoinitiator is chosen from 2,4,6-trimethylbenzoyl-phenylphosphinate oxide (TPO-L), camphorquinone, 4,4′-bis(diethylamino)benzophenone, 4,4′-bis(diethylamino)benzophenone combined with N-phenylglycine (NPG), with ethyl-4-(dimethylamino)benzoate (EDB), with N-diisopropylethylamine (DIPEAN) or with 4-(dimethylamino)benzonitrile (DMABN), biacylphosphine oxide (BAPO), titanium bis(.eta.5-2,4-cylcopentadien-1-yl)-bis(2,6-difluoro-3-(1H-pyrrol-1-yl)-phenyl) (Irgacure 784), 1-[4-(2-hydroxyethoxy)-phenyl]-2-hydroxy-2-methyl-1-propane-1-one (Irgacure 2959), 2,4,6-trimethylbenzoyldiphenylphosphine oxide (TPO), 2,2-dimethoxyphenyl-2-acetophenone (DMPA), and the mixtures thereof.

According to an embodiment, the photoinitiator is at a concentration ranging from 0.1% to 2% by weight relative to the total weight of the composition.

According to an embodiment, the photopolymerisable resin is chosen from: urethane acrylate resins, urethane methacrylate resins, epoxy acrylate resins, and the mixtures thereof; preferably the photopolymerisable resin is chosen from urethane acrylate resins, urethane methacrylate resins, difunctional epoxy acrylate resins and the mixtures thereof; more preferably the photopolymerisable resin is chosen from: a urethane acrylate resin, a urethane methacrylate resin and the mixtures thereof; still more preferably, the photopolymerisable resin is selected from aliphatic urethane acrylate resins, hydrophobic urethane acrylate resins, aromatic urethane acrylate resins, polyether urethane acrylate resins, aliphatic urethane methacrylate resins, hydrophobic urethane methacrylate resins, aromatic urethane methacrylate resins, polyether urethane methacrylate resins, and the mixtures thereof.

According to an embodiment, the photopolymerisable resin is at a concentration ranging from 10% to 94.9% by weight relative to the total weight of the composition.

The invention also relates to a composition according to the invention, for use thereof for the adhesion of non-mineralised biological tissues to one another; for the adhesion of a material to a non-mineralised biological tissue; for the adhesion of a glue or a substance to the surface of a non-mineralised biological tissue; as a surgical sealant on a non-mineralised biological tissue; to close or seal the orifices created by a thread suture or by a staple or by a tissue resection in a non-mineralised biological tissue; in order to close and orifice, an incision or a tear in a non-mineralised biological tissue; as a haemostatic in order to stop bleeding on a non-mineralised biological tissue, as a dressing on a non-mineralised biological tissue in order to cover and protect a wound; in order to reinforce a non-mineralised biological tissue; in order to prevent the formation of lesions on a non-mineralised biological tissue; in order to fix and stabilise a non-mineralised biological tissue; and/or for the treatment of skin lesions.

According to an embodiment, the composition for use thereof according to the invention, comprises placing the composition in contact with the non-mineralised biological tissue to be treated, preferably by spreading; and the photopolymerisation of said composition.

The present invention also relates to a method for preparing a polymerisable composition for use as an adhesive for non-mineralised biological tissues, comprising a step of mixing:
at least one polymerisable monomer comprising an acrylate, methacrylate, acrylamide or methacrylamide function and not comprising a phosphate or phosphonate function;
at least one photoinitiator; and
at least one photopolymerisable resin;
   said mixture not comprising a monomer comprising a phosphate or phosphonate function on the one hand and an acrylate, methacrylate, acrylamide or methacrylamide function on the other hand.

According to an embodiment, the weight of the polymerisable monomer varies from 5% to 60% by weight, relative to the total weight of said mixture; and the weight of the photoinitiator varies from 0.1% to 5% by weight, relative to the total weight of said mixture.

The present invention also relates to a kit comprising the composition of the invention as previously described.

According to an embodiment, the kit further comprises a radiation source.

### Definitions

In the present invention, the terms below are defined as follows:
“Polymerisable composition” refers to a composition comprising one or more components which can be polymerised in order to form a polymer.
“Polymerisable monomer” refers to a monomer for which polymerisation can be initiated by a physical or chemical initiator.
“Photoinitiator” refers to a compound which, when added to a monomer, can initiate photopolymerisation by creating reactive species (e.g. free radicals, cations, anions) under the effect of (UV or visible) radiation.
“Polymerisable resin” refers to a reactive oligomer of high molecular weight (generally greater than 450 g/mol, preferably greater than 1000 g/mol) which is able to be polymerised. In the present invention, the polymerisable resin is a photopolymerisable resin, in other words for which the polymerisation can be initiated under the effect of radiation.
“Biological tissues” refers to an assembly of similar cells of the same origin, grouped into clusters, networks or (fibre) bundles. A biological tissue forms a functional assembly, in other words its cells contribute to the same function. The biological tissue are assembled to form organs. In the present invention, the expression “biological tissues” also encompasses organs in a general manner.
“Non-mineralised biological tissues” refers to biological tissue with the exclusion of bone tissue, bones and teeth. Non-mineralised biological tissues are generally soft tissues.
The term “pharmaceutically acceptable” referring to a substance or a composition, means that the substance or composition are compatible with one another and/or not harmful to the subject, preferably a human being, to which the substance or composition is administered. In particular, they do not produce any undesirable allergic or other reaction when administered to the subject. For human administration, the compositions must meet the standards of sterility, pyrogenicity, general safety and purity required by the regulatory bodies, such as the Food and Drug Administration (FDA) or the European Medicines Agency (EMA).
“Subject” relates to a warm-blooded animal, preferably a mammal, more preferably a human. Preferably, the subject is a patient, in other words a subject who expects to receive, or who receives medical care, or who is or will be the subject of a medical procedure.
“From [lower value] to [higher value]” and other similar expressions define a numerical range which includes (in other words encompasses) both the upper value and the lower value. Moreover, any range that is defined in the present application must be interpreted as including an explicit disclosure of the narrowest corresponding range described by the expression “between [lower value] and [upper value]”, wherein both the upper value and lower value are excluded from the numerical range (in other words not encompassed).
The term “comprises” is intended to signify that the composition according to the invention includes the cited elements. This term also encompasses compositions comprising only the cited elements, to the exclusion of any other (composition “consisting of” the cited elements).

### DETAILED DESCRIPTION

### Polymerisable Composition

The present invention therefore relates to a polymerisable composition, for use as an adhesive for biological tissues, in particular for non-mineralised biological tissues, enabling, in particular, the adhesion of biological tissues to one another, the adhesion of a material to a biological tissue, the formation of a surgical sealant, or the formation of a skin dressing.

The invention relates, in particular, to polymerisable compositions for use as an adhesive for biological tissues, in particular for non-mineralised biological tissues, comprising:
at least one polymerisable monomer comprising an acrylate, methacrylate, acrylamide or methacrylamide function and not comprising a phosphate or phosphonate function;
at least one initiator of polymerisation, preferably a photoinitiator; and
at least one photopolymerisable resin.

The polymerisation of the composition of the invention makes it possible to form the targeted adhesive and to obtain adhesion properties for the resulting system.

The term “adhesive for biological tissues”, refers to an adhesive for use on biological tissues, in particular a medical use on biological tissues. The components of such an adhesive and of the polymerisable composition enabling it to be formed must therefore be by nature compatible with the biological tissue and in particular the medical uses. In particular, it must not cause any harmful effect (i.e. an undesirable allergic or other reaction) on the biological tissues concerned.

The composition of the invention is, in particular, intended to be used for non-mineralised biological tissues. This assumes that once applied and polymerised, said composition must make it possible to obtain an adhesive having physicochemical properties compatible with non-mineralised biological tissues, in other words soft tissues. In particular, the following properties must be obtained:
good flexibility of the adhesive, suitable for use on soft biological tissue, in order to protect the tissue from the exterior while ensuring wearing comfort (absorption of impacts, resistance and resilience when faced with large deformations);
good stability over time under the action of physiological liquids (e.g. blood, exudates, transpiration), in order, in particular, to maintain the adhesion properties in a humid environment and so as not to cause deformation on contact with physiological liquids in order to maintain the mechanical protection and adhesion to the biological tissue; and
preventing the accumulation of transpiration or exudates.

In the context of the present invention, when the concentrations of the various components of the composition according to the invention are indicated in percentages, these are the percentage by weight of said component relative to the total weight of said composition.

### Polymerisable Monomers

The composition of the invention comprises a polymerisable monomer comprising an acrylate, methacrylate, acrylamide or methacrylamide function, and not comprising a phosphate or phosphonate function. In the present invention, this type of monomer will be designated by the expression “non-phosphated polymerisable monomer”.

According to an embodiment, the non-phosphated polymerisable monomer is selected from the following monomers: acrylic acid (AA), tert-butyl acrylate (tBuA), 2-hydroxyethyl methacrylate (HEMA), methacrylic acid (MA), lauryl acrylate (LA), lauryl methacrylate (LMA), 2-ethoxyethyl methacrylate (2EEMA), di(ethylene glycol) ethyl ether acrylate (DEGEA), 2-phenyloxyethyl acrylate (2PEA), 2-ethylhexyl methacrylate (2EHMA), n-butyl acrylate (nBuA), isobornyl acrylate (IBOA), isobornyl methacrylate (IBOMA), cyclic trimethylolpropane formal acrylate (CTFA), 3,3,5 trimethyl cyclohexanol methacrylate, tert-butyl methacrylate (tBuMA), methyl acrylate (MeA), methyl methacrylate (MMA), 2-ethylhexyl acrylate (2EHA), 2-(dimethylamino)ethyl acrylate (DAEA), 3-sulfopropyl acrylate potassium salt (SAPS), 3,3-dimethacrylic acid (DAA), crotonic acid (CA), triethylene glycol methyl ether methacrylate (TEGMEMA), 2-phenyloxyethyl methacrylate (2PEMA), 2-hydroxyethyl acrylate (HEA), 3-(trimethoxysilyl) propyl methacrylate, ethyl acrylate (EtA), cyclohexyl methacrylate, 3-hydroxypropyl acrylate, alpha-bromoethyl acrylate, alpha-chlorocthyl acrylate, chloromethyl methacrylate, 2-bromoethyl, 2-naphtyl methacrylate, paratolyl acrylate, parachlorophenyl methacrylate, metabromophenyl acrylate, 2,4,6-tribromophenyl acrylate, parachlorobenzyl methacrylate, metamethoxybenzyl methacrylate, paracthylbenzyl acrylate, 1,6-hexanediol dimethacrylate, neopentylglycol diacrylate, thiodiethylene-glycol dimethacrylate, bisphenol A ethoxyl diacrylate, bisphenol A ethoxyl dimethacrylate, pentaerythritol triacrylate, glyceryl triacrylate, dipentaerythritol pentaacrylate, trimethylolpropane triacrylate, tris(2-hydroxyethyl) isocyanurate trimethacrylate, trimethylolpropane polyoxyethylene triacrylate, urethane acrylate, urethane methacrylate, bis(4-methacryloylthiophenyl) sulfide, ethyleneglycol acrylate, polyethyleneglycol acrylate, ethyleneglycol methacrylate, polyethyleneglycol methacrylate, cthyleneglycol diacrylate, polyethyleneglycol diacrylate, ethyleneglycol dimethacrylate, polyethyleneglycol dimethacrylate, and the mixtures thereof.

According to an embodiment, the non-phosphated polymerisable monomer is selected from the following monomers: acrylic acid (AA), tert-butyl acrylate (tBuA), 2-hydroxyethyl methacrylate (HEMA), methacrylic acid (MA), lauryl acrylate (LA), lauryl methacrylate (LMA), 2-ethoxyethyl methacrylate (2EEMA), di(ethylene glycol) ethyl ether acrylate (DEGEA), 2-phenyloxyethyl acrylate (2PEA), 2-ethylhexyl methacrylate (2EHMA), n-butyl acrylate (nBuA), isobornyl acrylate (IBOA), isobornyl methacrylate (IBOMA), cyclic trimethylolpropane formal acrylate (CTFA), 3,3,5 trimethyl cyclohexanol methacrylate (TMCHMA), tert-butyl methacrylate (tBuMA), methyl acrylate (MeA), methyl methacrylate (MMA), 2-ethylhexyl acrylate (2EHA), 2-(dimethylamino)ethyl acrylate (DAEA), 3-sulfopropyl acrylate potassium salt (SAPS), 3,3-dimethacrylic acid (DAA), crotonic acid (CA), triethylene glycol methyl ether methacrylate (TEGMEMA), 2-phenyloxyethyl methacrylate (2PEMA), 2-hydroxyethyl acrylate (HEA), 3-(trimethoxysilyl) propyl methacrylate, and the mixtures thereof.

According to an embodiment, the non-phosphated polymerisable monomer is selected from the following monomers: tert-butyl acrylate (tBuA), hydroxyethyl methacrylate (HEMA), acrylic acid (AA), lauryl acrylate (LA), isobornyl acrylate (IBOA), isobornyl methacrylate (IBOMA), 3,3,5 trimethyl cyclohexanol methacrylate (TMCHMA), and the mixtures thereof.

According to an embodiment, the non-phosphated polymerisable monomer is a mixture of two or more of the monomers listed above. In particular, the non-phosphated polymerisable monomer is a mixture of acrylic acid and another monomer listed above.

According to an embodiment, the non-phosphated polymerisable monomer has a molar mass ranging from 50 g·mol⁻¹ to 500 g·mol⁻¹, preferably from 70 g·mol⁻¹ to 400 g·mol⁻¹.

According to an embodiment, the non-phosphated polymerisable monomer is present in the composition of the invention at a concentration ranging from 5% to 60% by weight relative to the total weight of the composition, preferably from 5% to 50% by weight, preferably from 5% to 40% by weight. According to an embodiment, the non-phosphated polymerisable monomer is present in the composition of the invention at a concentration ranging from 10% to 40% by weight, preferably from 20% to 40% by weight, more preferably from 25% to 35% by weight. According to another embodiment, the non-phosphated polymerisable monomer is present in the composition of the invention at a concentration ranging from 5% to 30% by weight, more preferably from 10% to 20% by weight.

The polymerisation of the non-phosphated polymerisable monomer present in the composition of the invention can be initiated by a physical or chemical initiator. According to a preferred embodiment, the polymerisation is initiated under the effect of radiation, preferably radiation having a wavelength ranging from 300 nm to 520 nm, preferably from 350 nm to 520 nm. The monomers used in the composition of the invention have the advantage of polymerising rapidly even at low to moderate levels of irradiation, preferably ranging from 5 mW/cm² to 250 mW/cm², preferably from 10 mW/cm² to 100 mW/cm², preferably from 10 mW/cm² to 50 mW/cm². These low to moderate levels of irradiation are particularly suitable for use on biological tissues, because they do not emit UV C. Moreover, these low to moderate levels of irradiation have the advantage of being accessible with small radiation sources, as opposed to industrial radiation sources which are large and have high irradiance (>10 W/cm²).

According to an embodiment, the composition according to the invention does not comprise any monomer comprising, on the one hand, a phosphate or phosphonate function and on the other hand a methacrylate, acrylate, acrylamide or methacrylamide function. In the present invention, this type of monomer can be designated by the expression “phosphated polymerisable monomer”. According to an embodiment, the composition according to the invention does not comprise a polymerisable monomer selected from glycerol phosphate dimethacrylate, ethylene glycol methacrylate phosphate, polyethylene glycol methacrylate phosphate, methacryloyloxydecyl hydrogen phosphate, the methacryloyloxyethyl hydrogen phosphate, glycerol monomethacrylate phosphate, triethylene glycol monomethacrylate phosphate, methacryloyloxypropyl phosphate, methacryloyloxyhexyl phosphate, methacrylated aminoethyl phosphoric acid, bis(glyceryl dimethacrylate) phosphate, and the mixtures thereof. According to an embodiment, the composition according to the invention does not comprise a phosphated polymerisable monomer selected from CAS [14206-25-8], [14235-57-5], [86242-61-7], [932019-41-6], [1980781-17-6], [60161-88-8], [87243-97-8], [1980048-95-0], [918802-80-9], [63411-25-6], [1980064-07-0], [22432-83-3], [1980781-08-5], [252210-28-9], [1114567-37-1], [80730-17-2], [518991-74-7], [87243-96-7], [1980062-84-7], [518991-75-8], [252210-30-3], [22432-84-4], [727415-30-7], [727415-31-8], [784139-89-5] or [1194231-98-5] and the mixtures thereof. According to an embodiment, the composition according to the invention does not comprise a phosphated polymerisable monomer of formula I as described in WO2020/169681, in particular 10-MDP (C14H2706P, CAS number [85590-00-7]) or MEP (C12H19OgP, CAS number [32435-46-4]).

According to an embodiment, the composition according to the invention does not comprise any monomer for which the polymerisation can be initiated solely by contact with water, the humidity of the biological tissues and/or the ambient humidity. According to an embodiment, the composition according to the invention does not comprise any monomer of the cyanoacrylate family.

### Photopolymerisable Resins

The composition of the invention comprises a photopolymerisable resin. Advantageously, the presence of a photopolymerisable resin in the composition of the invention gives the adhesive obtained after polymerisation optimum flexibility properties for the targeted applications, in particular for adhesion of non-mineralised biological tissues, while limiting the exothermy of the polymerisation reaction of the composition to an acceptable level. An acceptable exothermy corresponds to a temperature where the polymerisation of the composition does not cause burns the tissue on which the composition is polymerised, nor any burning sensation. Preferably, the exothermy is controlled in order to ensure a polymerisation temperature less than 60° C.

Furthermore, the presence of the photopolymerisable resin in the composition of the invention contributes to providing the adhesive that is obtained after polymerisation with good stability over time under the action of physiological liquids and good resistance to water, and to preventing the accumulation of transpiration or exudates.

According to an embodiment, the photopolymerisable resin used in the composition of the invention is a resin which forms a flexible material after polymerisation.

According to an embodiment, the photopolymerisable resin is chosen from urethane acrylate resins, urethane methacrylate resins, epoxy acrylate resins, and the mixtures thereof.

According to an embodiment, the photopolymerisable resin is a urethane acrylate resin or a urethane methacrylate resin. According to a particular embodiment, the photopolymerisable resin is a urethane acrylate resin.

According to an embodiment, the urethane resin is selected from aliphatic urethane acrylate resins, hydrophobic urethane acrylate resins, aromatic urethane acrylate resins, polyethers urethane acrylate resins, aliphatic urethane methacrylate resins, hydrophobic urethane methacrylate resins, aromatic urethane methacrylate resins, polyether urethane methacrylate resins, and the mixtures thereof. According to an embodiment, the urethane resin is selected from aliphatic urethane acrylate resins, hydrophobic urethane acrylate resins, aromatic urethane acrylate resins, polyether urethane acrylate resins, and the mixtures thereof.

According to an embodiment, the aliphatic urethane acrylate resin is selected from Allnex Ebecryl 9907®, Allnex Ebecryl 230®, Allnex Ebecryl 250®, Allnex Ebecryl 8315® (tetraacrylate), Allnex Ebecryl 4491®, Allnex Ebecryl 1271®, and the mixtures thereof. According to an embodiment, the aromatic urethane acrylate resin is Allnex Ebecryl 210®. According to an embodiment, the hydrophobic urethane acrylate resin is Dymax Bomar BRC-843S®. According to an embodiment, the polyether urethane acrylate resin is the Dymax Bomar BR-3641AJ®.

According to an embodiment, the photopolymerisable resin is selected from Allnex Ebecryl 9907®, Allnex Ebecryl 230®, Allnex Ebecryl 250®, Allnex Ebecryl 8315® (tetraacrylate), Allnex Ebecryl 4491®, Allnex Ebecryl 1271®, Allnex Ebecryl 210®, Dymax Bomar BRC-843S®, Dymax Bomar BR-3641AJ®, and the mixtures thereof.

According to a particular embodiment, the photopolymerisable resin is Allnex Ebecryl 9907®.

According to an embodiment, the photopolymerisable resin is an epoxy acrylate resin; preferably a difunctional epoxy acrylate resin; more preferably the difunctional epoxy acrylate resin is chosen from: bisphenol A epoxy diacrylate resins, for example Allnex Ebecryl 3708®, epoxidised soybean oil acrylate resins, for example Rahn Genomer 2312®, and the mixtures thereof.

According to another embodiment, the photopolymerisable resin is a difunctional epoxy acrylate resin, in particular a bisphenol A epoxy diacrylate resin, for example Allnex Ebecryl 3708®.

According to an embodiment, the composition of the invention comprises at least 10% by weight photopolymerisable resin, relative to the total weight of the composition. According to an embodiment, the composition of the invention comprises from 10% to 94.9% by weight photopolymerisable resin, preferably from 50% to 94.9%. According to an embodiment, the composition of the invention comprises from 65% to 94.75% by weight, photopolymerisable resin. According to an embodiment, the composition of the invention comprises 65%, 68%, 69%, 79%, 89% and 94.75% by weight, photopolymerisable resin.

The polymerisation of the photopolymerisable resin can be initiated under the effect of radiation. Preferably, said radiation has a wavelength ranging from 300 nm to 520 nm, preferably from 350 nm to 520 nm. Preferably said UV radiation has an irradiance power ranging from 5 mW/cm² to 250 mW/cm², preferably from 10 mW/cm² to 100 mW/cm², preferably from 10 mW/cm² to 50 mW/cm².

### Photoinitiators

The composition of the invention comprises a polymerisation initiator, preferably a photoinitiator.

According to an embodiment, the photoinitiator is capable of inducing a polymerisation under the effect of radiation having a wavelength ranging from 300 nm to 520 nm, preferably from 350 nm to 520 nm.

According to an embodiment, the photoinitiator is chosen from: 2,4,6-trimethylbenzoyl-phenylphosphinate oxide (TPO-L), camphorquinone, 4,4′-bis(diethylamino)benzophenone, 4,4′-bis(diethylamino)benzophenone combined with N-phenylglycine (NPG), with ethyl-4-(dimethylamino)benzoate (EDB), with N-diisopropylethylamine (DIPEAN) or with 4-(dimethylamino)benzonitrile (DMABN), biacylphosphine oxide (BAPO), titanium bis(.eta.5-2,4-cylcopentadien-1-yl)-bis(2,6-difluoro-3-(1H-pyrrol-1-yl)-phenyl) (Irgacure 784), 1-[4-(2-hydroxyethoxy)-phenyl]-2-hydroxy-2-methyl-1-propane-1-one (Irgacure 2959), 2,4,6-trimethylbenzoyldiphenylphosphine oxide (TPO), 2,2-dimethoxyphenyl-2-acetophenone (DMPA), and the mixtures thereof.

According to a particular embodiment, the photoinitiator is TPO-L.

According to an embodiment, the photoinitiator is present in the composition of the invention at a concentration ranging from 0.1% to 5% by weight, relative to the total weight of the composition, preferably from 0.1% to 2% by weight, preferably from 0.25% to 2% by weight, more preferably from 0.5% to 2% by weight.

Unexpectedly, the photoinitiator plays a role in the adhesion properties after polymerisation, that can vary according to the quantity and type of photoinitiator used.

The use of a thermal or redox polymerisation initiator is not excluded from the scope of the present invention. The redox initiators that can be used include, in particular, the dibenzoyl peroxide/amine (trimethylaniline) pair.

### Particular Compositions

According to a particular embodiment, the composition of the invention comprises:
from 5% to 30% by weight, relative to the total weight of the composition, of a non-phosphated polymerisable monomer;
from 0.25% to 5% by weight of a photoinitiator; and
from 65% to 94.75% by weight of a photopolymerisable resin.

According to a particular embodiment, the composition of the invention comprises:
from 10% to 30% by weight, relative to the total weight of the composition, of a non-phosphated polymerisable monomer selected from tert-butyl acrylate (tBuA), hydroxyethyl methacrylate (HEMA), acrylic acid (AA), lauryl acrylate (LA), isobornyl acrylate (IBOA), isobornyl methacrylate (IBOMA), and 3,3,5 trimethyl cyclohexanol methacrylate (TMCHMA);
from 0.25% to 5% by weight TPO-L; and
from 65% to 94.75% by weight aliphatic urethane acrylate resin, such as Allnex Ebecryl 9907®.

According to a particular embodiment, the composition of the invention comprises:
from 10% to 30% by weight, relative to the total weight of the composition, of a non-phosphated polymerisable monomer selected from tert-butyl acrylate (tBuA), hydroxyethyl methacrylate (HEMA), acrylic acid (AA), lauryl acrylate (LA), isobornyl acrylate (IBOA), isobornyl methacrylate (IBOMA), and 3,3,5 trimethyl cyclohexanol methacrylate (TMCHMA);
from 0.25% to 5% by weight TPO-L; and
from 65% to 94.75%, preferably from 65% to 89.75%, by weight of a photopolymerisable resin chosen from the aliphatic urethane acrylate resins such as Allnex Ebecryl 9907®; difunctional epoxy acrylate resins such as the epoxy acrylate resin Allnex Ebecryl 3708® (corresponding to a modified epoxy bisphenol A diacrylate resin) or the epoxy acrylate resin Rahn Genomer 2312® (corresponding to an epoxidised soybean oil acrylate resin), and the mixtures thereof.

According to an embodiment, the composition of the invention further comprises a pharmaceutically acceptable vehicle. According to another embodiment, the composition of the invention contains no solvent.

### Properties of the Compositions

According to an embodiment, the composition of the invention is in liquid form.

According to an embodiment, the composition of the invention has a viscosity less than 120 Pa·s at 20° C., preferably less than 107 Pa·s at 20° C., preferably less than 55 Pa·s at 20° C. The viscosity of the composition can be measured, in particular, by a rotary viscometer, a cone and plate viscometer, a falling ball viscometer, a glass capillary viscometer, a flow cup viscometer or by extrusion under pressure, preferably according to standard NF EN 12092.

The adjustment of the viscosity of the composition makes it possible, in particular, to control the thickness of the layer of composition deposited on the biological tissue before its polymerisation, according to the intended use for the adhesive formed after polymerisation. For example, a thin layer, with a thickness less than or equal to 0.5 mm, is preferred for adhesion of a material to a biological tissue or for adhesion of a glue or a substance to the surface of a biological tissue. A thicker layer, of thickness greater than or equal to 1 mm, is preferred for forming a dressing on a biological tissue in order to cover and protect a wound.

According to an embodiment, the composition according to the invention is not a hydrogel.

### Kit

The present invention also relates to a kit, or set of parts, comprising a composition according to the invention and a radiation source.

Preferably, the radiation source of the set of parts can emit radiation suitable for polymerisation and/or for assisting the polymerisation and/or accelerating the polymerisation of components of the composition.

In the context of the present invention, the term “radiation source” refers to any artificial means capable of producing radiation of wavelength ranging from 300 nm to 520 nm, preferably from 350 nm to 520 nm. Preferably, said UV radiation has an irradiance power ranging from 5 mW/cm² to 250 mW/cm², preferably from 10 mW/cm² to 100 mW/cm², preferably from 10 mW/cm² to 50 mW/cm². Advantageously, these levels of irradiance can be obtained by lightweight radiation sources, suitable for use by the general public, as opposed to industrial radiation sources which are cumbersome and have high irradiance (>10 W/cm²). According to an embodiment, the radiation source is an LED UV source.

### Method for Preparing a Polymerisable Composition

The invention also relates to a method for preparing a polymerisable composition according to the invention. In particular, the invention relates to a method for preparing a polymerisable composition for use as an adhesive for non-mineralised biological tissues, said method comprising or consisting of a step of mixing:
at least one polymerisable monomer comprising an acrylate, methacrylate, acrylamide or methacrylamide function and not comprising a phosphate or phosphonate function, as previously described;
at least one photoinitiator, as previously described; and
at least one photopolymerisable resin, as previously described; and
   said mixture not comprising a monomer comprising a phosphate or phosphonate function on the one hand and an acrylate, methacrylate, acrylamide or methacrylamide function on the other hand.

According to an embodiment, the weight of the polymerisable monomer in the mixture represents from 5% to 60% of the total weight of said mixture.

According to an embodiment, the weight of the photoinitiator in the mixture represents from 0.1% to 5% of the total weight of said mixture.

### Use of the Polymerisable Compositions

The invention also relates to the use of polymerisable compositions of the invention as an adhesive for biological tissues, in particular for non-mineralised biological tissues.

In particular, the polymerisable compositions of the invention can be used on biological tissues, in particular on non-mineralised biological tissues, as surgical adhesive, surgical sealant, or skin dressing.

More particularly, the present invention relates to the use of polymerisable compositions of the invention, on biological tissues, in particular non-mineralised biological tissues:
for the adhesion of the biological tissues to one another,
for the adhesion of a material to a biological tissue,
for the adhesion of a glue or of a substance to the surface of a biological tissue,
as a surgical sealant, to close or seal the orifices created by a thread suture or by a staple or by a tissue resection (haemostasis, aerostasis, lymphostasis, for example),
for closing an orifice, an incision or a tear in a biological tissue,
as a haemostatic in order to stop bleeding, alone or in combination with conventional haemostasis techniques such as suturing, compression or electrocoagulation,
as a dressing on a biological tissue in order to cover and protect a wound,
in order to reinforce a biological tissue,
in order to fix and stabilise a biological tissue,
for the treatment of skin lesions.

The invention therefore relates to a polymerisable composition according to the invention for use thereof on biological tissues, in particular on non-mineralised biological tissues, as surgical adhesive, surgical sealant, or skin dressing.

The present invention also relates to a polymerisable composition according to the invention for use thereof as a surgical adhesive for the adhesion of biological tissues to one another, for adhesion of a material to a biological tissue, for adhesion of a glue or substance to the surface of a biological tissue, as a surgical sealant, to close or seal the orifices created by a thread suture or by staple or by a tissue resection, in order to close an orifice, an incision or a tear in a biological tissue, as a haemostatic in order to stop bleeding, as a dressing on a biological tissue in order to cover and protect a wound, to reinforce a biological tissue, in order to fix and stabilise a biological tissue, for the treatment of skin lesions. The biological tissues are preferably non-mineralised biological tissues.

The present invention also relates to a composition according to the invention for use thereof as a surgical adhesive for the adhesion of non-mineralised biological tissues to one another, for example for producing a suture, in particular a skin wound suture.

The present invention also relates to a composition according to the invention for use thereof as a surgical adhesive for the adhesion of a material to a non-mineralised biological tissue, for example for making a dressing or a surgical drape adhere to the skin.

The present invention also relates to a composition according to the invention for use as a surgical adhesive for the adhesion of a glue or a substance to the surface of a non-mineralised biological tissue, for example in order to reinforce the properties of a surgical glue.

The present invention also relates to a composition according to the invention for use thereof as a surgical sealant, in particular to close or seal the orifices created by a thread suture or by a staple or by a tissue resection, in a non-mineralised biological tissue.

The present invention also relates to a composition according to the invention for use thereof as a surgical sealant in order to close an orifice, an incision or a tear in a non-mineralised biological tissue.

The present invention also relates to a composition according to the invention for use thereof as a haemostatic in order to stop bleeding.

The present invention also relates to a composition according to the invention for use thereof in order to form a dressing on a non-mineralised biological tissue in order to cover and protect a wound.

The present invention also relates to a composition according to the invention for use thereof as a surgical adhesive in order to reinforce a non-mineralised biological tissue.

The present invention also relates to a composition according to the invention for use thereof as a surgical adhesive in order to prevent the formation of lesions on a tissue, in particular a skin tissue; for example in order to prevent the formation of blisters.

The present invention also relates to a composition according to the invention for use thereof as a surgical adhesive in order to fix and stabilise a non-mineralised biological tissue.

The present invention also relates to a composition according to the invention for use thereof for the treatment of skin lesions.

It has been shown, in particular, that the compositions according to the invention had adhesive capacities and safety greater than the compositions of the prior art. These compositions are therefore particularly suitable for use on the skin as a dressing for injuries and/or for the treatment of all skin lesions.

### Method for Using the Compositions of the Invention

The present invention also relates to a method for using the polymerisable composition of the invention, comprising the following steps:
(i) placing the biological tissue to be treated in contact with a composition according to the invention;
(ii) optionally, allowing the composition to penetrate into said tissue; and
(iii) inducing the polymerisation of said composition.

According to an embodiment, in step (i), the placing of the biological tissue to be treated in contact with the composition can be carried out by spreading, spraying or by placing in contact with the help of a pre-coated material, such as a pre-coated patch. According to an embodiment, the contacting is carried out by spreading.

According to an embodiment, step (i) aims to totally or partially coat the biological tissue to be treated.

According to an embodiment, step (ii) is optional. The penetration time of the composition into the tissue before its polymerisation can range from 0 to 5 minutes.

According to a preferred embodiment, step (iii) is carried out with the assistance of UV radiation or visible light. The properties of the radiation used, in particular its power and its wavelength, are adjusted to the components of the composition, in particular to the nature of the polymerisable monomer and to the nature of the polymerisation initiator. According to an embodiment, the radiation used to induce the polymerisation has a wavelength ranging from 300 nm to 520 nm, preferably from 350 nm to 520 nm. According to an embodiment, the radiation used to induce the polymerisation has an irradiance power ranging from 5 mW/cm² to 250 mW/cm², preferably from 10 mW/cm² to 100 mW/cm², preferably from 10 mW/cm² to 50 mW/cm².

According to an embodiment, the composition of the invention gives a biocompatible system after polymerisation.

The compositions according to the invention make it possible to obtain excellent adhesion after polymerisation, both for surgical applications as well as for a the level of skin adhesion suitable for application by the general public.

The concentrations of components of the composition of the invention enable rapid initiation and polymerisation in several tens of seconds, preferably polymerisation in less than 1 minute, without causing tissue burns.

Furthermore, the combined presence of a photopolymerisable resin, with the polymerisable monomers used and the photoinitiator, gives the bonding obtained properties of flexibility, good stability over time under the action of physiological liquids (e.g. blood, exudates, transpiration), prevents the accumulation of transpiration or exudates and provides good resistance to water.

The method according to the invention is preferably non-invasive. The term “non-invasive” signifies that the method according to the invention does not comprise any surgical step consisting of accessing the tissue to be treated. Thus, the method according to the invention is implemented on a directly accessible biological tissue (e.g. the skin) or a tissue made accessible beforehand by other methods.

The method according to the invention is preferably a non-invasive method in order to cover and protect a skin lesion. The method according to the invention is preferably a non-invasive method for bringing the edges of a skin wound closer together. The term “skin” designates a location situated on the skin, the lips or the oral mucosa.

Alternatively, the method according to the invention is a method for the adhesion of a material to a biological tissue, for the adhesion of biological tissues to one another, for the adhesion of a glue or a substance to the surface of a biological tissue, surgical sealing, to close or seal the orifices created by a thread suture or by a staple or by a tissue resection (haemostasis, aerostasis, lymphostasis, for example), for closing an orifice, an incision or a tear in a biological tissue, in order to stop bleeding, in order to cover and protect a wound, in order to reinforce a biological tissue, in order to prevent the formation of lesions on a biological tissue, or in order to fix and stabilise a biological tissue.

### EXAMPLES

The present invention will be understood more clearly on reading the following examples which illustrate the invention non-restrictively.

### Example 1: Evaluation of Compositions According to the Invention

Aim: Various polymerisable compositions according to the invention were prepared and evaluated for their use as an adhesive for biological tissues.

### Compositions:

Compositions according to the invention were prepared with the following commercially available compounds:
photoinitiator: TPO-L (ethyl phenyl(2,4,6-trimethylbenzoyl)phosphinate);
   photopolymerisable resin: aliphatic urethane acrylate resin Allnex Ebecryl 9907®; epoxy acrylate resin Allnex Ebecryl 3708® (corresponding to a bisphenol A epoxy diacrylate resin); epoxy acrylate resin Genomer 2312® (corresponding to an epoxidised soybean oil acrylate resin) from RAHN;
   non-phosphated polymerisable monomers: tert-butyl acrylate (tBuA), hydroxyethyl methacrylate (HEMA), acrylic acid (AA), lauryl acrylate (LA), isobornyl acrylate (IBOA), isobornyl methacrylate (IBOMA), 3,3,5 trimethyl cyclohexanol methacrylate (TMCHMA).

The compositions listed in Table 1 were prepared by mixing various components. In Table 1, the proportions of the various components are given as percentage by weight relative to the total weight of the composition.

Composition 15 (without photopolymerisable resin) is not part of the invention and is presented in Table 1 as a control composition.

TABLE 1

| Composition | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| tBuA | 30 | | | | | | | | | | | | | | |
| HEMA | | 30 | | | | | | | | | | | | | 74 |
| LA | | | 30 | | | | 10 | 20 | 30 | 30 | 30 | | | | |
| IBOA | | | | 30 | | | | | | | | | | | |
| IBOMA | | | | | 30 | | | | | | | | | | |
| TMCHMA | | | | | | 30 | | | | | | | | | |
| AA | | | | | | | | | | | | 5 | | 5 | 25 |
| TPO-L | 1 | 1 | 1 | 1 | 1 | 11 | 1 | 1 | 0.5 | 2 | 5 | 0.25 | 1 | 0.25 | 1 |
| Ebecryl 9907 ® | 69 | 69 | 69 | 69 | 69 | 69 | 89 | 79 | 69.5 | 68 | 65 | 94.75 | | | |
| Ebecryl 3708 ® | | | | | | | | | | | | | | 94.25 | |
| Genomer 2312 ® | | | | | | | | | | | | | 69 | | |

The emission spectrum of the LED UV source used for polymerising the formulations is centred on 395 nm and used at an irradiance of 20 mW/cm².

### Evaluated Criteria:

the polymerisation time: a rapid photopolymerisation is targeted, preferably with a duration of less than 1 minute, in order in particular to be able to control the bonding and for a facilitated use, while being carried out at low to moderate levels of irradiation (preferably from 5 mW/cm² to 250 mW/cm²) which are suitable for use on biological tissues (i.e. no emission of UV C) and accessible with lightweight radiation sources, as opposed to industrial radiation sources;
the conversion rate: a high final conversion rate is targeted, preferably a conversion rate greater than 90%, for an optimised polymerisation and avoiding the presence of too high a proportion of non-polymerised compounds in the final adhesive, which could exhibit toxicity;
exothermy: the exothermy of the polymerisation reactions must be limited for comfort of the subject and must not cause tissue burns;
adhesion to the tissues and holding of the bond over time (in particular the holding in vivo): the adhesion and holding over time must be sufficiently good to durably accompany the tissue healing process and to limit the number of dressing changes, which cause irritation or lesions of the peri-lesion tissue;
resistance to water: good resistance to water (and to biological fluids) is sought in order to maintain the adhesion property in a humid environment, to avoid deformation on contact with water/physiological fluids in order to maintain mechanical protection and the adhesion to the biological tissue, and to durably protect the skin in the event of contact or immersion in an aqueous medium (daily housework activities, hand washing, shower, nautical activities, etc.) while avoiding maceration of the tissue under the bonding;
the flexibility of the network formed: the network formed must have a flexibility suitable for use on soft biological tissue in order to protect the tissue from the exterior and to ensure wearing comfort (absorption of impacts, resistance and resilience when faced with large deformations);
the mechanical strength of the network formed, including resistance to crumbling and tearing.

### Methodology:

Qualitative evaluation of in vivo adhesion is carried out by a qualitative skin evaluation on the hand of volunteer testers (N=1 to 5 tests). A qualitative adhesion scale was developed (Table 2) taking into account the following evaluation criteria for adhesion and holding:
adhesion at t=0;
anchoring in the surface layer of the skin;
holding over time; and
resistance to stress.

TABLE 2

| | |
|---|---|
| 0 | no adhesion at t = 0, no anchoring |
| 1 | adhesion at t = 0, very weak anchoring, detachment in several |
| | minutes, no resistance to stress |
| 2 | adhesion at t = 0, weak anchoring, holding <1 hour, no |
| | resistance to stress |
| 3 | adhesion at t = 0, weak anchoring, holding 2 hours to 4 hours, |
| | weak resistance to stress |
| 4 | adhesion at t = 0, weak anchoring, holding 4 hours to 8 hours, |
| | weak resistance to stress |
| 5 | adhesion at t = 0, medium anchoring, holding 4 hours to 8 hours, |
| | medium resistance to stress |
| 6 | adhesion at t = 0, medium anchoring, holding 8 hours to 12 hours, |
| | medium resistance to stress |
| 7 | adhesion at t = 0, medium anchoring, holding 12 hours to 24 hours, |
| | good to medium resistance to stress |
| 8 | adhesion at t = 0, good anchoring, holding 24 hours to 36 hours, |
| | good resistance to stress |
| 9 | adhesion at t = 0, good anchoring, holding 36 hours to 48 hours, |
| | very good resistance to stress |
| 10 | adhesion at t = 0, excellent anchoring, holding >48 hours, |
| | excellent resistance to stress |

The polymerisation time and the final conversion rate are evaluated by kinetic monitoring of photopolymerisation in ATR-IR. The disappearance of IR absorption bands characteristic of acrylates is quantified under 395 nm UV LED radiation, with an irradiance of 17 mW/cm².

The exothermy of photopolymerisation is qualitatively evaluated in vivo by a qualitative skin evaluation on the hand of volunteer testers. The scale of feeling is described as follows:
0 no heat felt;
+ weak exothermy felt;
++ moderate exothermy felt;
+++ high exothermy felt;
x pain during the polymerisation.

The flexibility is evaluated qualitatively by two means. On the one hand, the resistance to curvature of polymer films obtained by polymerisation under 395 nm LED UV radiation for 1 minute at an irradiance of 17 mW/cm², is qualitatively evaluated. On the other hand, the flexibility is qualitatively evaluated in vivo via an appreciation of the wearing comfort on the hand of volunteer testers. The following flexibility scale is used:
0 glassy brittle film, uncomfortable, can cause irritation at the edges of the bonding;
+ deformable but rigid film, uncomfortable;
++ flexible film, comfortable;
+++ very flexible film, comfortable, perfectly follows the movements of the biological tissue.

The mechanical strength is qualitatively evaluated by two means. On the one hand, polymer films obtained by polymerisation under 395 nm LED UV radiation for 1 minute at an irradiance of 17 mW/cm², are subjected to a manual tearing stress. On the other hand, the resistance to crumbling is qualitatively evaluated by manual shearing of the surface and edges of the plate. The following scale for mechanical strength is used:
0 hydrogel texture, crumbles and tears easily under stress;
+ fragile and sensitive to tearing;
++ medium sensitivity to tearing;
+++ resistant to tearing;
++++ very resistant to tearing.

The sensitivity to water is qualitatively evaluated by two means. First, polymer films obtained by polymerisation under 395 nm LED UV radiation for 1 minute at an irradiance of 17 mW/cm² are immersed in water at 40° C. for 24 hours to 72 hours and macroscopic observations (bleaching, deformation, softening, weakening) are collected. Second, the sensitivity to water is qualitatively evaluated by a hand washing test of volunteer testers. The following scale for sensitivity to water is used:
0 no change;
+ low to moderate swelling;
++ bleaching/high swelling;
+++ bleaching/high swelling/deformation/loss of mechanical strength.

++++ Total physical alteration (destruction of the sample)

### Results

The results obtained from the various compositions tested are reported in Table 3.

TABLE 3

| | Holding | Polymerisation | | | | Mechanical | Sensitivity |
|---|---|---|---|---|---|---|---|
| Composition | in vivo | time | | Exothermy | Flexibility | strength | to water |
| 1 | 4 | <10 | s | 0 | +++ | +++ | 0 |
| 2 | 5 | ~20 | s | 0 | ++ | ++++ | 0 |
| 3 | 5 | <10 | s | 0 | +++ | ++ | 0/slight blanching |
| 4 | 5 | ~20 | s | + | +++ | +++ | 0 |
| 5 | 5 | ~30 | s | + | ++ | +++ | 0 |
| 6 | 5 | ~20 | s | + | ++ | +++ | 0 |
| 7 | 7 | <1 | min | 0 | +++ | +++ | 0/slight blanching |
| 8 | 7 | <1 | min | + | +++ | ++ | 0/slight blanching |
| 9 | 6 | <30 | s | 0 | +++ | ++ | 0/slight blanching |
| 10 | 6 | <10 | s | ++ | +++ | ++ | 0/slight blanching |
| 11 | 7 | <10 | s | ++ | +++ | ++ | 0/slight blanching |
| 12 | 7 | <1 | min | + | +++ | ++++ | 0 |

The conversion rates obtained for all the tested compositions of the invention above are greater than 90%.

The compositions of the invention have the desired properties of adhesion to a biological tissue as well as the desired properties of flexibility and compatibility suitable for non-mineralised biological tissue. Furthermore, the compositions of the invention make it possible to obtain bonds having good stability over time.

By contrast, the results show that a composition not comprising photopolymerisable resin, does not enable both good adhesion and flexibility properties to be obtained. Moreover, the polymer film from this composition is very sensitive to water. More specifically, the polymer film from composition 15 was highly altered and its immersion in water at 40° C. for 24 hours to 72 hours led to the destruction of this polymer film.

1-14. (canceled)

15. A polymerisable composition, for use as an adhesive for non-mineralised biological tissues, comprising:
5% to 60% by weight, relative to the total weight of the composition, of a polymerisable monomer comprising an acrylate, methacrylate, acrylamide or methacrylamide function and not comprising a phosphate or phosphonate function;
0.1% to 5% by weight of a photoinitiator; and
a photopolymerisable resin;
said composition not comprising a monomer comprising a phosphate or phosphonate function on the one hand and an acrylate, methacrylate, acrylamide or methacrylamide function on the other hand.

16. The composition according to claim 15, wherein the polymerisable monomer is chosen from: acrylic acid (AA), tert-butyl acrylate (tBuA), 2-hydroxyethyl methacrylate (HEMA), methacrylic acid (MA), lauryl acrylate (LA), lauryl methacrylate (LMA), 2-ethoxyethyl methacrylate (2EEMA), di(ethylene glycol) ethyl ether acrylate (DEGEA), 2-phenyloxyethyl acrylate (2PEA), 2-ethylhexyl methacrylate (2EHMA), n-butyl acrylate (nBuA), isobornyl acrylate (IBOA), isobornyl methacrylate (IBOMA), cyclic trimethylolpropane formal acrylate (CTFA), 3,3,5 trimethyl cyclohexanol methacrylate, tert-butyl methacrylate (tBuMA), methyl acrylate (MeA), methyl methacrylate (MMA), 2-ethylhexyl acrylate (2EHA), 2-(dimethylamino)ethyl acrylate (DAEA), 3-sulfopropyl acrylate potassium salt (SAPS), 3,3-dimethacrylic acid (DAA), crotonic acid (CA), triethylene glycol methyl ether methacrylate (TEGMEMA), 2-phenyloxyethyl methacrylate (2PEMA), 2-hydroxyethyl acrylate (HEA), 3-(trimethoxysilyl) propyl methacrylate, ethyl acrylate (EtA), cyclohexyl methacrylate, 3-hydroxypropyl acrylate, alpha-bromoethyl acrylate, alpha-chloroethyl acrylate, chloromethyl methacrylate, 2-bromoethyl methacrylate, 2-naphthyl methacrylate, paratolyl acrylate, parachlorophenyl methacrylate, metabromophenyl acrylate, 2,4,6-tribromophenyl acrylate, parachlorobenzyl methacrylate, metamethoxybenzyl methacrylate, paraethylbenzyl acrylate, 1,6-hexanediol dimethacrylate, neopentylglycol diacrylate, thiodiethylene-glycol dimethacrylate, bisphenol A ethoxyl diacrylate, bisphenol A ethoxyl dimethacrylate, pentaerythritol triacrylate, glyceryl triacrylate, dipentaerythritol pentaacrylate, trimethylolpropane triacrylate, tris(2-hydroxyethyl) isocyanurate trimethacrylate, trimethylolpropane polyoxyethylene triacrylate, urethane acrylate, urethane methacrylate, bis(4-methacryloylthiophenyl) 17.sulfide, ethyleneglycol acrylate, polyethyleneglycol acrylate, ethyleneglycol methacrylate, polyethyleneglycol methacrylate, ethyleneglycol diacrylate, polyethyleneglycol diacrylate, ethyleneglycol dimethacrylate, polyethyleneglycol dimethacrylate, and the mixtures thereof.

17. The composition according to claim 16, wherein the polymerisable monomer is chosen from: acrylic acid (AA), tert-butyl acrylate (tBuA), 2-hydroxyethyl methacrylate (HEMA), methacrylic acid (MA), lauryl acrylate (LA), lauryl methacrylate (LMA), 2-ethoxyethyl methacrylate (2EEMA), di(ethylene glycol) ethyl ether acrylate (DEGEA), 2-phenyloxyethyl acrylate (2PEA), 2-ethylhexyl methacrylate (2EHMA), n-butyl acrylate (nBuA), isobornyl acrylate (IBOA), isobornyl methacrylate (IBOMA), cyclic trimethylolpropane formal acrylate (CTFA), 3,3,5 trimethyl cyclohexanol methacrylate, tert-butyl methacrylate (tBuMA), methyl acrylate (MeA), methyl methacrylate (MMA), 2-ethylhexyl acrylate (2EHA), 2-(dimethylamino)ethyl acrylate (DAEA), 3-sulfopropyl acrylate potassium salt (SAPS), 3,3-dimethacrylic acid (DAA), crotonic acid (CA), triethylene glycol methyl ether methacrylate (TEGMEMA), 2-phenyloxyethyl methacrylate (2PEMA), 2-hydroxyethyl acrylate (HEA), 3-(trimethoxysilyl) propyl methacrylate, and the mixtures thereof.

18. The composition according to claim 15, wherein the polymerisable monomer is at a concentration ranging from 10% to 40% by weight relative to the total weight of the composition.

19. The composition according to claim 15, wherein the photoinitiator is chosen from 2,4,6-trimethylbenzoyl-phenylphosphinate oxide (TPO-L), camphorquinone, 4,4′-bis(diethylamino)benzophenone, 4,4′-bis(diethylamino)benzophenone combined with N-phenylglycine (NPG), with ethyl-4-(dimethylamino)benzoate (EDB), with N-diisopropylethylamine (DIPEAN) or with 4-(dimethylamino)benzonitrile (DMABN), biacylphosphine oxide (BAPO), titanium bis(cta.5-2,4-cylcopentadien-1-yl)-bis(2,6-difluoro-3-(1H-pyrrol-1-yl)-phenyl) (Irgacure 784), 1-[4-(2-hydroxyethoxy)-phenyl]-2-hydroxy-2-methyl-1-propane-1-one (Irgacure 2959), 2,4,6-trimethylbenzoyldiphenylphosphine oxide (TPO), 2,2-dimethoxyphenyl-2-acetophenone (DMPA), and the mixtures thereof.

20. The composition according to claim 15, wherein the photoinitiator is at a concentration ranging from 0.1% to 2% by weight relative to the total weight of the composition.

21. The composition according to claim 15, wherein the photopolymerisable resin is chosen from: urethane acrylate resins, urethane methacrylate resins, epoxy acrylate resins, and the mixtures thereof.

22. The composition according to claim 15, wherein the photopolymerisable resin is at a concentration ranging from 10% to 94.9% by weight relative to the total weight of the composition.

23. A composition according to claim 15, for use thereof for the adhesion of non-mineralised biological tissues with one another; for the adhesion of a material to a non-mineralised biological tissue; for the adhesion of a glue or a substance to the surface of a non-mineralised biological tissue; as surgical sealant on a non-mineralised biological tissue; to close or seal the orifices created by a thread suture or by a staple or by a tissue resection in a non-mineralised biological tissue; in order to close an orifice, an incision or a tear in a non-mineralised biological tissue; as a haemostatic in order to stop bleeding on a non-mineralised biological tissue, as a dressing on a non-mineralised biological tissue in order to cover and protect a wound; in order to reinforce a non-mineralised biological tissue; in order to prevent the formation of lesions on a non-mineralised biological tissue, in order to fix and stabilise a non-mineralised biological tissue; and/or for the treatment of skin lesions.

24. The composition for use thereof according to claim 23, wherein the composition is to be placed in contact with the non-mineralised biological tissue to be treated; and the photopolymerisation of said composition.

25. A method for preparing a polymerisable composition for use as an adhesive for non-mineralised biological tissues, comprising a step of mixing:
at least one polymerisable monomer comprising an acrylate, methacrylate,
acrylamide or methacrylamide function and not comprising a phosphate or phosphonate function;
at least one photoinitiator; and
at least one photopolymerisable resin;
said mixture not comprising a monomer comprising a phosphate or phosphonate function on the one hand and an acrylate, methacrylate, acrylamide or methacrylamide function on the other hand.

26. The method according to claim 25, wherein the weight of the polymerisable monomer varies from 5% to 60% by weight, relative to the total weight of said mixture; and the weight of the photoinitiator varies from 0.1% to 5% by weight, relative to the total weight of said mixture.

27. A kit comprising a device containing the polymerisable composition according to claim 15.

28. The kit according to claim 27, further comprising a radiation source.

29. The composition according to claim 21, wherein the photopolymerisable resin is chosen from the urethane acrylate resins, urethane methacrylate resins, difunctional epoxy acrylate resins and the mixtures thereof; more preferably the photopolymerisable resin is chosen from: a urethane acrylate resin, a urethane methacrylate resin and the mixtures thereof; still more preferably, the photopolymerisable resin is selected from aliphatic urethane acrylate resins, hydrophobic urethane acrylate resins, aromatic urethane acrylate resins, polyether urethane acrylate resins, aliphatic urethane methacrylate resins, hydrophobic urethane methacrylate resins, aromatic urethane methacrylate resins, polyether urethane methacrylate resins, and the mixtures thereof.

30. The composition according to claim 29, wherein the photopolymerisable resin is chosen from a urethane acrylate resin, a urethane methacrylate resin and the mixtures thereof.

31. The composition according to claim 30, wherein the photopolymerisable resin is selected from aliphatic urethane acrylate resins, hydrophobic urethane acrylate resins, aromatic urethane acrylate resins, polyether urethane acrylate resins, aliphatic urethane methacrylate resins, hydrophobic urethane methacrylate resins, aromatic urethane methacrylate resins, polyether urethane methacrylate resins, and the mixtures thereof.

32. A method comprising the application of the composition according to claim 15, for the adhesion of non-mineralised biological tissues with one another; for the adhesion of a material to a non-mineralised biological tissue; for the adhesion of a glue or a substance to the surface of a non-mineralised biological tissue; as surgical sealant on a non-mineralised biological tissue; to close or seal the orifices created by a thread suture or by a staple or by a tissue resection in a non-mineralised biological tissue; in order to close an orifice, an incision or a tear in a non-mineralised biological tissue; as a haemostatic in order to stop bleeding on a non-mineralised biological tissue, as a dressing on a non-mineralised biological tissue in order to cover and protect a wound; in order to reinforce a non-mineralised biological tissue; in order to prevent the formation of lesions on a non-mineralised biological tissue, in order to fix and stabilise a non-mineralised biological tissue; and/or for the treatment of skin lesions.

33. The method according to claim 32, comprising placing the composition in contact with the non-mineralised biological tissue to be treated; and the photopolymerisation of said composition.

34. The method according to claim 33, wherein the composition is placed in contact with the non-mineralised biological tissue to be treated by spreading.
